(19) United States
(12) Patent Application Publication
(10) Pub. No.: US 2026/0176329 A1
(43) Pub. Date: Jun. 25, 2026

(54) AMYLASE COMPOSITIONS FOR PRODUCING COLLAGEN PEPTIDES AND METHODS OF FORMING COLLAGEN PEPTIDES

(71) Applicant: Rochal Technologies, LLC, San Antonio, TX (US)

(72) Inventors: Rebecca E. McMAHON, San Antonio, TX (US); Ann Beal Salamone, Gainesville, FL (US); Nina Bionda, Ithaca, NY (US); Bradley J. Rodier, San Antonio, TX (US); Jonathan D. Belasco, San Antonio, TX (US); Tiffany Fawn Sanchez, Helotes, TX (US); Sergio Medina, San Antonio, TX (US); Fahimeh Tabatabaei, Boerne, TX (US)

(73) Assignee: Rochal Technologies, LLC, San Antonio, TX (US)

(21) Appl. No.: 19/416,925

(22) Filed: Dec. 11, 2025

Related U.S. Application Data
(60) Provisional application No. 63/733,543, filed on Dec. 13, 2024.

(51) Int. Cl. C07K 14/78; C12N 9/26; C12N 9/28; C12N 9/34; C12P 21/02

(52) U.S. Cl. CPC C07K 14/78; C12N 9/2417; C12N 9/2425; C12N 9/2428; C12P 21/02; C12Y 302/01001; C12Y 302/01002; C12Y 302/01003

(57) ABSTRACT

A composition for producing collagen low molecular weight peptides (10,000 Da or less) that utilizes the amylase family of enzymes. The amylases (α-, β-, γ-amylase) are noted for the cleavage of the α-glycosidic bonds of polysaccharides, yielding lower molecular weight carbohydrate/sugar fragments. It has been found that α-amylase is effective in reacting with animal tissues to yield hydrolyzed collagen peptides, which facilitate cell migration and proliferation.

### CROSS-REFERENCE TO RELATED APPLICATIONS

This application claims priority under 35 U.S.C. § 119 (e) to U.S. Provisional Application No. 63/733,543, filed on Dec. 13, 2024, the entire content of which is incorporated herein by reference.

### FIELD OF INVENTION

The present invention relates to compositions comprising denatured collagen and amylase to produce collagen peptides.

### BACKGROUND

Current compositions for producing low molecular weight collagen peptides, also known as hydrolyzed collagen, utilize acid, base, proteolytic enzymatic processes, or a combination of these processes to hydrolyze the protein collagen. Collagen is found in most animal tissue and is the most abundant protein found in the extracellular matrix of connective tissue (cartilage, bones, tendons, ligaments, skin) but is also found in corneas, blood vessels, dentin and muscle tissue. Approximately 25 to 35 wt-% of a mammal's protein content is collagen. Because of the high protein content of collagen, enzymatic processes for producing collagen peptides utilize protease sources, which may contain trace amounts of other enzymes (e.g., amylases, lipases). Proteases commonly used for enzymatic hydrolysis of collagen are endoproteases, such as serine endoproteases (trypsin and alcalase) and aspartyl endoproteases (pepsin), which hydrolyze internal amide bonds within a protein chain.

Of note, the extracellular matrix of connective tissue is complex and is known to haves two main components: (1) polysaccharide chains known as glycosaminoglycans (GAGs), which usually are covalently bound to proteins, and (2) fibrous proteins such as collagen, elastin, fibronectin and laminin.

Collagen-containing material may be derived from human, bovine, porcine, murine, avian, marine, other animal and combinations thereof. It may be derived from soft or hard tissues, and combinations thereof.

### SUMMARY OF THE INVENTION

It is an object of the amylase-containing compositions, also written as compositions, described herein to combine denatured collagen and an enzymatic formulation that cleaves α-1,4-glycosidic linkages in polysaccharides, glycoproteins and proteoglycans, to produce compositions containing collagen peptides with a molecular weight less than 10,000 Da.

It is a further object of the amylase-containing compositions described herein to provide an enzymatic formulation for digestion of denatured collagen-containing material utilizing the amylase family of enzymes.

It is a further object of the methods described herein to provide an enzymatic formulation for the digestion of denatured collagen-containing materials comprising the step of administering an effective amount of an amylase enzyme.

It is a further object of the amylase-containing compositions described herein to provide an enzymatic formulation containing enzymes for the digestion of denatured collagen-containing material utilizing α-amylase.

It is a further object of the amylase-containing compositions described herein to provide an enzymatic formulation containing enzymes for digestion of denatured collagen-containing material utilizing β-amylase.

It is a further object of the amylase-containing compositions described herein to provide an enzymatic formulation containing enzymes for digestion of denatured collagen-containing material utilizing γ-amylase.

It is a further object of the amylase-containing compositions described herein to provide an enzymatic formulation containing enzymes for digestion of denatured collagen-containing material utilizing enzymes from the amylase family, selected from combinations of α-amylase, with β-amylase, and γ-amylase.

It is a further object of the amylase-containing compositions described herein to provide for collagen-containing material digestion utilizing predominantly amylase, with a minority amount (20 wt % or less) of other families of enzymes.

It is a further object of the amylase-containing compositions described herein to provide for digestion of denatured collagen-containing material wherein the enzymatic formulation contains 20 wt-% or less of enzymes other than amylases.

It is a further object of the amylase-containing compositions described herein to provide for digestion of denatured collagen-containing material wherein the enzymatic formulation contains 20 wt-% or less of enzymes such as proteases, chondroitinases, hyaluronidases, lipases, glycosidases, heparanases, dermatanases, pullulanases, N-acetylglucosaminidase, lactases, phospholipases, transglycosylases, esterases, thioester hydrolyases, sulfatases, escharases, nucleases, phosphatases, phosphodiesterases, mannanases, mannosidases, isoamylases, lyases, inulinases, keratinases, tannases, pentosanases, glucanases, arabinosidases, pectinases, cellulases, chitinases, xylanases, cutinases, pectate lyases, hemicellulases, combinations thereof, and the like.

It is a further object of the amylase-containing compositions described herein to provide for digestion of denatured collagen-containing material wherein the enzymatic formulation contains 20 wt-% or less of enzymes such as oxidases, peroxidases, glucose oxidases, catalases, oxidoreductases, phenoloxidases, laccases, lipoxygenases, isomerases, ligninases, combinations thereof, and the like.

It is an object of the amylase-containing compositions described herein to provide enzymatic digestion of denatured collagen-containing materials that is not based on peptide cleavage by proteases.

It is a further object of the amylase-containing compositions described herein to administer the enzymatic formulations on or within the denatured collagen-containing material.

It is a further object of amylase-containing compositions described herein to provide for digestion of denatured collagen-containing material utilizing amylase, wherein the amylase is applied in a hydrophilic or aqueous medium.

It is a further object of the amylase-containing compositions described herein to further comprise a surfactant capable of solubilizing, swelling, or hydrating the denatured collagen-containing material contained therein.

It is a further object of the amylase-containing compositions described herein to further comprise a hydrophilic polymer capable of increasing viscosity or causing gelation of the composition.

### DETAILED DESCRIPTION OF THE INVENTION

In this specification and the appended claims that follow, reference will be made to a number of terms that shall be defined to have the following meanings:

It must be noted that, as used in the specification and the appended claims, the singular forms “a”, “an” and “the” include plural references unless the context clearly dictates otherwise. Thus, for example, reference to “a surfactant” includes mixtures of two or more such surfactants, and the like.

As used herein, the term “about” indicates that a value can vary by up to +5%, +2%, or +1%.

As used herein, unless otherwise specified, “amylase-containing compositions” and “compositions” are equivalent and are used interchangeably to describe compositions that include denatured collagen and amylase. In some embodiments, the amylase-containing compositions include other ingredients (e.g., additives, actives, excipients, etc.).

As used herein, “denaturation” and “denature” have their standard meaning and include the loss of three-dimensional structure for biological macromolecules.

As used herein, the terms “glycosidic bond” and “glycosidic linkage” refer to a covalent bond that is formed between a carbohydrate molecule and another carbohydrate, amino acid, or lipid.

As used herein, the term “1,4-glycosidic linkages” are bonds that are normally formed between the carbon-1 on one sugar and the carbon-4 on another sugar moiety in a polysaccharide. An α-1,4-glycosidic bond is formed when the —OH group on carbon-1 is below the plane of the glucose ring. On the other hand, a β-1,4-glycosidic bond is formed when the —OH group is above the plane. For example, cellulose is formed of glucose molecules linked by β-1,4-glycosidic bonds, whereas starch is composed of α-1,4 glycosidic bonds.

As used herein, “α-amylase” includes naturally occurring α-amylases as well as recombinant α-amylases, wherein recombinant α-amylase means an α-amylase in which the DNA genetic sequence encoding the naturally occurring α-amylase is modified to produce a mutant DNA sequence that encodes the substitution, insertion or deletion of one or more amino acids in the α-amylase sequence compared to the naturally occurring α-amylase. Further, α-amylase has a starch dextrinizing activity of 250 to 250,000 U/gram.

As used herein, “amylolytic” is characterized by or capable of the enzymatic digestion of starch into dextrins and sugars, particularly by amylase.

As used herein, “non-proteolytic enzyme” has its standard meaning and includes enzymes that independently cleave (digest, break, hydrolyze) non-protein molecules into shorter fragments. Examples of non-proteolytic enzymes includes amylase, lipase, etc.

As used herein, “proteolytic enzyme” has its standard meaning and includes enzymes that independently cleave (digest, break, hydrolyze) the long chainlike polymer molecules of proteins into shorter fragments of peptides and, eventually, into their basic components of amino acids.

As used herein, the amount of enzyme utilized is expressed in weight percent and its activity is given in Units of activity per gram, where a “Unit” is defined as the amount of enzyme that catalyzes the conversion of 1 micromole of substrate per minute.

As used herein, “enzyme formulation” and “enzymatic formulation” refers only to the enzymatic components of the amylase-containing compositions described herein. In other words, the denatured collagen, additives, excipients, etc. present in the amylase-containing composition are not part of the enzymatic component.

As used herein, “surfactant” has its standard meaning and includes compounds that lower the surface tension (or interfacial tension) between two liquids or between a liquid and a solid and includes emulsifying agents, emulsifiers, detergents, wetting agents, and surface-active agents.

As used herein, “microemulsion” has its standard meaning and includes thermodynamically stable mixtures of oil, water (and/or hydrophilic compound), and surfactant. Microemulsions include three basic types: direct (oil dispersed in water, o/w), reverse (water dispersed in oil, w/o) and bi-continuous. Microemulsions are optically clear because the dispersed micelles have a diameter that is less than the wavelength of visible light (e.g., less than 380 nanometers, less than 200 nanometers, or less than 100 nanometers) in diameter. In the absence of opacifiers, microemulsions are optically clear, isotropic liquids.

As used herein, “reverse microemulsion” has its standard meaning and includes a microemulsion comprising a hydrophilic phase suspended in a continuous oil phase. A reverse microemulsion can include droplets of a hydrophilic phase (e.g., water, alcohol, or a mixture of both) stabilized in an oil phase by a reverse emulsion surfactant. In such instances, a hydrophilic active agent can be solubilized in the droplets. However, in other instances, the reverse microemulsion can be free of water and/or alcohol, and the hydrophilic active agent can be directly solubilized in the oil phase by the reverse emulsion surfactant.

As used herein, “hydrophilic” has its standard meaning and includes compounds that have an affinity to water and can be ionic or neutral or have polar groups in their structure that attract water. For example, hydrophilic compounds can be miscible, swellable, or soluble in water.

As used herein, “hydrophobic” has its standard meaning and refers to repelling water, being insoluble or relatively insoluble in water, and lacking an affinity for water. Hydrophobic compounds with hydrophilic substituents may form emulsions in water, with or without added surfactant.

As used herein, “amphoteric” refers to a mixture of cationic and anionic charges on a molecule or polymer in which overall charge is locally pH dependent, whereas “ampholytic” has an equal number of cationic and anionic charges over a broad pH range.

As used herein, “aqueous” compositions refer to a spectrum of water-based solutions including, but not limited to, homogeneous solutions in water with solubilized components, emulsified solutions in water stabilized by surfactants or hydrophilic polymers, and viscous or gelled homogeneous or emulsified solutions in water.

As used herein, an enzyme is “soluble” or “solubilized” if the amount of enzyme present in the solvent system is dissolved in the solvent system without the enzyme forming a precipitate or visible, swollen gel particles in solution.

As used herein, an “excipient” is a usually inert substance that forms a vehicle, such as a liquid, fluid, or gel, that solubilizes or disperses an enzyme or other added ingredients.

The compositions described herein comprise amylase (e.g., α-amylase) and denatured collagen-containing tissue. Unexpectedly, the compositions described herein produce collagen peptides (e.g., hydrolyzed collagen) with molecular weights of less than 10,000 Da. Because the hydrolyzation process is facilitated by amylase, the resulting collagen peptides are unlike those formed using acid, base, or other enzymes (i.e., protease).

In one aspect, a novel composition for enzymatic digestion of denatured collagen-containing material containing amylase is provided which composition comprises denatured collagen and an enzymatic formulation comprising at least 50 wt-% amylase. Although not necessary for practicing the invention, it is believed that the enzymatic digestion not only includes cleavage of glycosidic bonds in and/or between glycosaminoglycan polysaccharides and collagen fibrils, but surprisingly also includes cleavage of peptide bonds. It was unexpectedly found that amylase, an enzyme noted for cleavage of α-1,4-glycosidic bonds, such as in the catalyzed hydrolysis of starch into low molecular weight sugars, is effective in the digestion of denatured collagen-containing tissue. This discovery provides for a novel composition for high efficiency enzymatic digestion with an enzyme (amylase) that does not digest itself, as it is not a protease.

In some embodiments, an amylase-containing composition is provided that comprises denatured collagen; and an enzymatic formulation comprising at least 50 wt-% amylase based on the weight of total solids in the enzymatic formulation.

In some embodiments, the enzymatic formulation comprises at least 50 wt-% of α-amylase. In some embodiments, the enzymatic formulation comprises at least 60 wt-%, at least 70 wt-%, at least 80 wt-%, at least 90 wt-%, at least 99 wt-%, or at least 100 wt-% of α-amylase. In some embodiments, the amount of amylase in a non-proteolytic enzymatic component of the enzymatic formulation can be 100 wt %, at least 90 wt %, or at least 80 wt %, at least 70 wt %, at least 60 wt %, or at least 50 wt % of the enzymatic formulation.

In some embodiments, the enzymatic formulation comprises α-amylase, β-amylase, γ-amylase, or combinations thereof. In some embodiments, the enzymatic formulation comprises β-amylase. In some embodiments, the enzymatic formulation comprises γ-amylase.

In some embodiments, a weight ratio of non-proteolytic enzymes to proteolytic enzymes is at least 10:1. In some embodiments, a weight ratio of non-proteolytic enzymes to proteolytic enzymes is at least 15:1, at least 20:1 at least 40:1, at least 60:1, at least 80:1, or at least 100:1. If no proteolytic enzymes are present, the ratio is infinite, which reads on at least 10:1.

In some embodiments, a weight ratio of amylase to proteolytic enzymes is at least 10:1. In some embodiments, a weight ratio of amylase to proteolytic enzymes is at least 15:1, at least 20:1 at least 40:1, at least 60:1, at least 80:1, or at least 100:1. If no proteolytic enzymes are present, the ratio is infinite, which reads on at least 10:1.

In some embodiments, the enzymatic formulation comprises less than 0.1 wt-% of proteolytic enzymes based on the total solids in the enzymatic formulation. In some embodiments, the enzymatic formulation comprises less than 0.01% by weight or less than 0.001% by weight of proteolytic enzymes.

In some embodiments, the enzymatic formulation comprises up to 10 wt-% of proteolytic enzyme based on the total solids in the enzymatic formulation, or up to 5 wt-%, or up to 1 wt-%.

In some embodiments, the denatured collagen comprises one or more of denatured bovine collagen, denatured porcine collagen, denatured murine collagen, denatured avian collagen, denatured marine collagen or combinations thereof. In some embodiments, the denatured collagen comprises denatured bovine collagen. In some embodiments, the denatured collagen comprises denatured porcine collagen. In some embodiments, the denatured collagen comprises denatured murine collagen. In some embodiments, the denatured collagen comprises denatured avian collagen. In some embodiments, the denatured collagen comprises denatured marine collagen.

In some embodiments, the amylase-containing composition described herein comprises a keratolytic agent. In some embodiments, the amylase-containing composition comprises a keratolytic agent in an amount of up to 15 wt-% of the solids of the composition. In some embodiments, the amylase-containing composition described herein comprises a keratolytic agent in an amount of up to 10 wt-% of the solids of the composition. In some embodiments, the composition comprises a keratolytic agent in an amount of up to 5 wt-% of solids of the composition.

In some embodiments, the amylase-containing composition described herein can include a keratolytic agent in an amount of at least 0.01 wt-% of solids in the composition. In some embodiments, the amylase-containing composition comprises a keratolytic agent in an amount of at least 0.1 wt-%, at least 0.25 wt-%, or at least 0.5 wt-% of solids in the composition

In some embodiments, the keratolytic agent is selected from the group consisting of urea, salicylic acid and α-hydroxyacids, such as lactic acid, glycolic acid, citric acid, and combinations thereof. In some embodiments, the keratolytic agent comprises urea.

In some embodiments, the amylase-containing composition described herein can include one or more surfactants to enhance digestion. Suitable surfactants include, but are not limited to, cationic, anionic, nonionic, amphoteric and ampholytic surfactants. In some embodiments, the surfactants are nonionic and amphoteric surfactants. In some embodiments, the surfactant can be present in an amount ranging from 0% to 10 wt % based on the weight of the composition. In some embodiments, the surfactant can be present in an amount of at least 0.01 wt %, or at least 0.1 wt %, or at least 0.25 wt %, or at least 0.5 wt %, or at least 1 wt %, based on the total solids of the composition.

The surfactants can have an HLB (hydrophilic-lipophilic balance) value of 18 to 30 in order to maintain the enzyme catalytic structure in solution as well as not hindering the biocidal activity of any added antimicrobial agents. The high values of the HLB represent surfactants that are more hydrophilic than those with lower HLB values.

Suitable nonionic surfactants include, but are not limited to, the ethylene oxide/propylene oxide block copolymers of poloxamers, reverse poloxamers, poloxamines, and reverse poloxamines. Poloxamers and poloxamines are preferred, and poloxamers are most preferred. Poloxamers and poloxamines are available under the trade names Pluronic® and Tetronic®, respectively. Suitable Pluronic surfactants comprise but are not limited to Pluronic F38 having a HLB of 31, Pluronic F68 having a HLB of 29, Pluronic 68LF having a HLB of 26, Pluronic F77 having a HLB of 25, Pluronic F87 having a HLB of 24, Pluronic F88 having a HLB of 28, Pluronic F98 having a HLB of 28, Pluronic F108 having a HLB of 27, Pluronic F127 (also known as Poloxamer 407) having a HLB of 18-23, and Pluronic L35 having a HLB of 19. An exemplary poloxamine surfactant of this type is Tetronic 1107 (also known as Poloxamine 1107) having an HLB of 24.

In addition to the above, other neutral surfactants may be added to the amylase-containing composition, such as for example polyethylene glycol esters of fatty acids, e.g., coconut, polysorbate, polyoxyethylene or polyoxypropylene ethers of higher alkanes (C₁₂-C₁₈), polysorbate 20 available under the trademark Tween 20, polyoxyethylene (23) lauryl ether available under the trademark Brij 35, polyoxyethylene (40) stearate available under the trademark Myrj 52, and polyoxyethylene (25) propylene glycol stearate available under the trademark Atlas G 2612. Other neutral surfactants include nonylphenol ethoxylates such as nonylphenol ethoxylates, Triton X-100, Brij surfactants of polyoxyethylene vegetable-based fatty ethers, Tween 80, decyl glucoside, and lauryl glucoside.

Amphoteric surfactants suitable for use in the compositions described herein include materials of the type offered commercially under the trademark Miranol. Another useful class of amphoteric surfactants is exemplified by betaines, including cocoamidopropyl betaine, undecylenamidoalkylbetaine, and lauramidoalkylbetaine and sodium cocoamphoacetate. Amphoteric surfactants are very mild and have excellent dermatological properties, making them particularly suited for use in personal care and medical applications.

The amylase-containing composition described herein can include an antimicrobial agent. As used herein, an “antimicrobial agent” is defined as a substance that kills microorganisms or inhibits their growth or replication. Antimicrobial agents have a broad spectrum of activity against bacteria, fungi, viruses, protozoa and prions. Examples of antimicrobial agents useful in the composition described herein include biguanides, such as poly(hexamethylene biguanide hydrochloride) (PHMB), chlorhexidine and its salts, alexidine and its salts, povidone/iodine, cadexomer iodine, silver sulfadiazine, nanocrystalline silver, ionic silver, honey, dilute bleaching agents such as sodium hypochlorite and hypochlorous acid, hydrogen peroxide, organic peroxides such as benzoyl peroxide, alcohols such as ethanol and isopropanol, monoalkyl glycols, glycerol alkyl ethers, monoacyl glycerols, anilides such as triclocarban, bisphenols such as triclosan, chlorine compounds such as chlorine dioxide and N-chloramines, and quaternary ammonium compounds such as benzalkonium chloride, benzethonium chloride, cetyltrimethylammonium chloride, cetylpyridinium chloride, and alkyltrimethylammonium bromides, as well as miconazole, clotrimazole, ketoconazole, fluconazole, crystal violet, amphotericin B, tea tree oil, and the like. Biguanides, such as PHMB, are useful in the composition described herein.

The interaction of low molecular weight PHMB polycation (molecular weight ˜2,400 Daltons) with amylase, a negatively charged, high molecular weight protein (molecular weight 55,000 Daltons) at physiological pH can generate a protein-polyelectrolyte complex of PHMB ionically interacted with amylase. In tissue, PHMB may be released from the protein-polyelectrolyte complex in a continuous release fashion as a function of the amount of low molecular weight cation in bodily fluids (such as sodium, potassium, calcium and magnesium ions) displacing the cationic PHMB ionic interaction with anionic amylase sites.

In some embodiments, the amylase-containing composition described herein is an aqueous composition with a pH in the range of 6.5 to 7.5. In some embodiments, the amylase-containing composition is an aqueous composition with a pH in the range of 3.0 to 6.5. In some embodiments, the amylase-containing composition is an aqueous composition with a pH in the range of 7.5 to 10.

In some embodiments, the amylase-containing composition described herein may further include a penetration enhancer. In some embodiments, the penetration enhancer can be present in an amount less than 5 wt % based on the total solids of the composition. In some embodiments, the penetration enhancer can be present in an amount of at least 0.01 wt %, or at least 0.1 wt %, or at least 0.25 wt %, or at least 0.5 wt %, or at least 1 wt-%, based on the total solids of the composition.

A penetration enhancer may be utilized to enhance efficacy of the enzymatic formulation. Penetration enhancers that can be incorporated into the composition include, but are not limited to, fatty acids such as branched and linear C₆-C₁₈ saturated acids, unsaturated acids, such as C₁₄ to C₂₂, oleic acid, cis-9-octadecenoic acid, linoleic acid, linolenic acid, fatty alcohols, such as saturated C₈-C₁₈ terpenes, such as d-limonene, α-pinene, 3-carene, menthone, fenchone, pulegone, piperitone, eucalyptol, chenopodium oil, carvone, menthol, α-terpineol, terpinen-4-ol, carveol, limonene oxide, pinene oxide, cyclopentane oxide, triacetin, cyclohexane oxide, ascaridole, 7-oxabicylco [2,2,1]heptane, 1,8-cineole, glycerol monoethers, glycerol monolaurate, glycerol monooleate, isostearyl isostearate, isopropyl myristate, isopropyl palmitate, isopropyl lanolate, pyrrolidones, such as N-methyl-2-pyrrolidone, 1-ethyl-2-pyrrolidone, 5-methyl-2-pyrrolidone, 1,5-dimethyl-2-pyrrolidone, 2-pyrrolidone-5-carboxylic acid, N-hexyl-2-pyrrolidone, N-lauryl-2-pyrrolidone, 1-dodecylazacycloheptan-2-one, 4-decyloxazolidin-2-one, N-dodecylcaprolactam, and 1-methyl-3-dodecyl-2-pyrrolidone N-butyl-N-dodecylacetamide, N,N-bisdodecylacetamide, N-cycloheptyl-N-dodecylacetamide and N,N-bispropyldodecanamide, urea, 1-dodecylurea, 1,3-didodecylurea, 1,3-diphenyl urea, dimethyl sulfoxide, decylmethyl sulfoxide, tetradecylmethyl sulfoxide, cyclodextrins, and combinations thereof. Also effective penetration enhancers include 1-alkyl-2-piperidinones, 1-alkyl-2-azacycloheptanones, such as 1-dodecyazacycloheptan-2-one, 1,2,3-alkanetriols, such as 1,2,3-nonanetriol, 1,2-alkanediols, 2-(1-alkyl)-2-methyl-1,3-dioxolanes, oxazolidinones, such as 4-decyloxazolidin-2-one, N,N-dimethylalkanamides, 1,2-dihydroxypropyl alkanoates, such as 1,2-dihydroxypropyl decanoate, 1,2-dihydroxypropyl octanoate, sodium deoxycholate, trans-3-alken-1-ols, cis-3-alken-1-ols, and trans-hydroxyproline-N-alkanamide-C-ethylamide, and combinations thereof. In some embodiments, the penetration enhancers can include hydrophobic esters isopropyl myristate, isopropyl palmitate, or combinations thereof.

In some embodiments, the denatured collagen is present in an amount ranging from 50 wt % to 99.99 wt-% based on a total solids weight of the composition (i.e., volatile or non-volatile solvents are not included in this calculation). In some embodiments, the denatured collagen is present in an amount ranging from 90 to 99 wt-% based on a total solids weight of the composition.

In some embodiments, the enzymatic formulation is present in an amount ranging from 0.01 wt % to 10 wt % based on a total solids weight of the composition. In some embodiments, the enzymatic formulation is present from 1 to 5 wt % based on a total solids weight of the composition.

In some embodiments, a ratio of denatured collagen to enzymatic formulation can range from 10:1 to 100:1.

The enzymatic formulations described herein can contain various types of the enzyme, amylase. Amylases (α-, β-, γ-amylase) are a family of enzymes that preferentially hydrolyze the α-glycosidic bonds of polysaccharides, yielding lower molecular weight carbohydrate/sugar fragments. In some embodiments, α-amylase is used as the amylase. Amylase occurs naturally in humans and other mammals, and it is also found in plants, bacteria and fungi. Frequently, naturally occurring amylase is present as a minority constituent in combination with other enzymes, such as protease.

The physical behavior of skin tissue is determined primarily by an extensive extracellular matrix (ECM). The ECM is composed of an interlocking mesh of fibrous proteins and glycosaminoglycans (GAGs) and glycoproteins. The GAGs are carbohydrate polymers and are usually attached to ECM proteins, forming proteoglycans. In skin, type I collagen is the main protein component of the ECM and key proteoglycan components are decorin and versican. Presumably, these core proteins bind to the surface of type I collagen fibrils, which provide mechanical strength to skin. Proteoglycan binding is required for appropriate assembly of collagen fibrils in the ECM and inhibits cleavage of collagen fibrils by matrix metalloproteases. Proteoglycans are composed of a protein backbone to which one or several GAG chains are covalently bonded. Four different classes of glycosaminoglycans exist in vertebrates, chondroitin sulfate, dermatan sulfate, keratan sulfate, and heparan sulfate/heparin. Hyaluronan (hyaluronic acid), a GAG containing β (1-4) linkages, is one of the chief components of the extracellular matrix, and it contributes significantly to cell proliferation and migration. However, unlike the other glycosaminoglycans, hyaluronan does not attach to proteins to form proteoglycans but binds and retains water molecules and fills the gaps between collagen fibrils. The GAGs are attached to a serine residue of the core protein by both glycosidic β-1,4-(primarily by chondroitin sulfate, dermatan sulfate) and α-1,4-bonds (primarily by heparan sulfate/heparin).

Whereas α-amylases catalyze the hydrolysis of internal α-(1-4)-linkages of carbohydrates as their main reaction, some α-amylases, particularly saccharifying amylases, catalyze transfer reactions in addition to hydrolysis (Keller 2013). These α-amylases are capable of transferring glycoside residues to low molecular weight alcohols as well as to water, a property related to the transferase activity of the glycosidases. It is not known whether such a trans-glycosylation process is operative in the digestion of denatured collagen-containing tissue as related to the amylase-containing compositions described herein.

Amylase is a digestive enzyme that aids in the cleavage of bonds in sugar residues in polysaccharides. It is found in two primary types in the human body: salivary amylase and pancreatic amylase. In saliva, salivary amylase is responsible for the breakdown of starch and glycogen into glucose, maltose, and dextrin. Pancreatic amylase further degrades starches in the digestive system. Pancreatic enzyme mixtures are a common source for naturally occurring proteases. Pancreatic enzyme mixtures include proteases as the majority constituent, along with lipases and amylase.

Of the three forms of amylase, α-amylase (also called 1,4-α-D-glucan glucanohydrolase) is an endoamylase that is found in all living organisms. It functions in a random manner by a multiple-attack mechanism on starch, glycogen and related polysaccharides and oligosaccharides with α-1,4-glycosidic linkages, ultimately yielding glucose and maltose, as well as larger oligosaccharides. The α-amylase hydrolyzes 1,4-α-D-glucosidic linkages in polysaccharides that contain 3 or more 1,4-α-linked D-glucosidic units. However, α-amylase cannot hydrolyze α-1,6-bonds in glycogen and amylopectin.

β-Amylase (also called 1,4-α-D-glucan maltohydrolase) and γ-amylase (also called (amyloglucosidase, glucan 1,4-α-glucosidase, and glucoamylase) are exoamylases that are found in plants and microorganisms. Like α-amylase, β-amylase cannot hydrolyze 1,6-α-bonds. The β-amylase enzyme acts on the same substrates as α-amylase, but β-amylase removes successive maltose units from the non-reducing end of the polysaccharide. γ-Amylase releases β-D-glucose successively from the non-reducing end of the polysaccharide chains.

Unlike α-amylase and β-amylase, various forms of γ-amylase can hydrolyze 1,6-α-D-glucosidic bonds when the next bond in the sequence is a 1,4-bond, and some preparations can hydrolyze 1,6- and 1,3-α-D-glucosidic bonds in other polysaccharides. Combinations of the amylase enzymes are used in various preparations, such as food production, sweeteners, starch saccharification, brewing and distilling industries.

Calcium and chloride ions are essential for the activity of α-amylase. One Ca²⁺ is tightly bound by each α-amylase molecule, facilitating the proper conformation for hydrolytic activity. Chloride ions have been regarded as natural activators of the enzyme. Excess calcium stabilizes α-amylase towards heat. Catalytic activity is optimum at a temperature range between 40° C. and 45° C. and a pH of 7.0-7.5.

In some embodiments, the amylase is from a source selected from bacteria, fungi, or animal. In some embodiments, the amylase is from a fungal source. In some embodiments, the amylase is from an animal. In some embodiments, the amylase is from a mammal.

In some embodiments, the amylase is from a bacterial source. In some embodiments, the bacterial source is Bacillus spp, or Aspergillus spp. In some embodiments, the bacterial source is Bacillus licheniformis, Bacillus amyloliquefaciens, Bacillus subtilis, or Bacillus stearothermophilus. In some embodiments, the bacterial source is Bacillus amyloliquefaciens.

In some embodiments, the activity of amylase can range from 250 Units to 250,000 Units per gram.

The amylase-containing compositions described herein can be formulated into a liquid, gel, powder, paste, ointment, lotion, emulsion, or microemulsion, and combinations and the like. The enzymatic formulation can be delivered to or contacted with the denatured collagen on or within the denatured collagen or a combination of both.

The composition can include one or more excipients that are compatible with the composition. Examples of excipients include, but are not limited to, water, normal saline (isotonic saline), Dulbecco's phosphate buffered saline (DPBS), phosphate buffered saline (PBS), saline solutions containing added calcium chloride, Ringer's solution, glycerin, propylene glycol, ethanol, isopropanol, butane-1,3-diol, liquid poly(alkylene glycol) s (e.g., poly(ethylene glycol), methyl ether-terminated poly(ethylene glycol), poly(ethylene glycol-block-propylene glycol-block-ethylene glycol)), water-soluble liquid silicone polyethers, and water-insoluble media, such as, isopropyl myristate, isopropyl palmitate, mineral oil, dimethicone, and petrolatum.

In some embodiments, excipients can be present in an amount ranging from 0% to 99.9 wt % based on the total weight of the amylase-containing composition. In some embodiments, excipients can be present in an amount less than 5 wt %.

In some embodiments, the composition can also include wetting agents, buffers, gelling agents, and emulsifiers. Other excipients include, but are not limited to, water-based buffers ranging in pH from 5.0-7.5, silicones, polyether copolymers, vegetable and plant fats and oils, essential oils, hydrophilic and hydrophobic alcohols, vitamins, monoglycerides, and penetration enhancement esters such as laurate esters, myristate esters, palmitate esters, and stearate esters.

In some embodiments, the enzymatic formulation is lyophilized to a dry powder. The lyophilized enzymatic formulation may be used in powder form, or the powder may be further formulated into solutions, creams, lotions, gels, sprays, foams, aerosols, films, or other formulations.

In some embodiments, a surfactant can be used to create enzymatic formulation emulsions, which facilitate compatibilization with organic solvents. Examples of organic solvents include, but are not limited to, non-inflammatory solvents, such as volatile silicone solvents and volatile alkanes to form water-in-oil or oil-in-water emulsions, reverse emulsions, miniemulsions (nanoemulsions), microemulsions, and reverse microemulsions. Non-inflammatory volatile silicone solvents include, but are not limited to low molecular weight polydimethylsiloxanes, such as hexamethyldisiloxane or octamethyltrisiloxane; low molecular weight cyclic siloxanes, such as hexamethylcyclotrisiloxane or octamethylcyclotetrasiloxane; linear, branched or cyclic alkanes, such as propane, butane, and isobutane (aerosols under pressure), pentane, hexane, heptane, octane, isooctane, and isomers thereof, petroleum distillates, and cyclohexane; and chlorofluorocarbons, such as, trichloromonofluoromethane, dichlorodifluoromethane, and dichlorotetrafluoroethane; fluorocarbons, such as tetrafluoroethane, heptafluoropropane, 1,1-difluoroethane, pentafluoropropane, perfluoroheptane, perfluoromethylcyclohexane; hydrofluoroalkanes, such as aerosols of 1,1,1,2,-tetrafluoroethane and 1,1,1,2,3,3,3-heptafluoropropane, combinations thereof and the like; and volatile gases under pressure, such as air, nitrous oxide, and liquid carbon dioxide; or a mixture thereof. As will be understood, when stored under high pressure, carbon dioxide can be present in the form of a liquid at room temperature. In some embodiments, the volatile solvent can be hexamethyldisiloxane, isooctane, or mixtures thereof. In some embodiments, the volatile solvent can be hexamethyldisiloxane. In some embodiments, solvents can be present in an amount ranging from 0% to 99.9 wt % based on the total weight of the amylase-containing composition.

In some embodiments, water-soluble polymers that are neutral in charge and are not enzymatically degradable by amylase can be used as viscosity builders within the composition. Examples of such viscosity builders include, but are not limited to, poly(ethylene oxide), poly(ethylene glycol), poly(vinyl alcohol), and poly(N-vinylpyrrolidone). Other viscosity builders useful in the composition described herein include, but are not limited to, neutral polysaccharides that have β-linkages between monosaccharide units, such as in methylcellulose, hydroxyethylcellulose, hydroxypropylcellulose, hydroxypropylmethylcellulose. Still other viscosity builders useful in the composition described herein include, but are not limited to, those that are anionic in charge, such as Carbomer and its salts, poly(acrylic acid) and it salts, and poly(methacrylic acid) and its salts. Still other viscosity builders are anionic polysaccharides that have β-linkages between monosaccharide units, such as carboxymethylcellulose. Such viscosity builders may be employed in amounts ranging from about 0.01 to about 50.0 weight percent of the amylase-containing composition for preparation of viscous gels or pastes. Viscosity builders can be present in amounts ranging from 0.1 to 45% by weight, from 0.5 to 25% by weight, or from 1.0 to 10.0% by weight of the amylase-containing composition.

In some embodiments, a keratolytic agent can also be added to the composition to aid in digesting keratolytic material present in the denatured collagen-containing material. For example, the keratolytic agent can promote the softening and peeling of the epidermis. Keratolytic agents useful in the composition as described herein include, but are not limited to, urea, salicylic acid and α-hydroxyacids, such as lactic acid, glycolic acid, and citric acid. In some embodiments, the keratolytic agent can be present in an amount ranging from 0% to 15 wt % based on the weight of solids in the composition. In some embodiments, essential oils can be present in an amount of at least 0.1 wt %, or at least 0.25 wt %, or at least 0.5 wt % based on the weight of solids in the amylase-containing composition

In some embodiments, the total amount of non-proteolytic enzyme in the enzymatic formulation can be at least 90 wt % to 100 wt % based on the total solids of the enzymatic formulation. In some embodiments, the non-proteolytic enzyme is an amylase. In some embodiments, the non-proteolytic enzyme is α-amylase.

In some embodiments, the amount of non-proteolytic enzymatic component in the enzymatic formulation can be 100 wt %, or at least 99.5 wt %, or at least 99 wt %, or at least 95 wt %, or at least 90 wt %, based on the solids in the formulation

In some embodiments, the amount of amylase in the enzymatic formulation can be 100 wt %, at least 90 wt %, at least 80 wt %, at least 70 wt %, at least 60 wt %, or at least 50 wt % based on the enzymatic formulation. In some embodiments, the amount of amylase in a non-proteolytic enzymatic component of the enzymatic formulation can be 100 wt %, at least 90 wt %, or at least 80 wt %, of the enzymatic formulation.

The amount of α-amylase can be at least 10 wt %, at least 20 wt %, at least 30 wt %, at least 40 wt %, at least 50 wt %, at least 60 wt %, at least 70 wt-%, at least 80 wt %, at least 90 wt %, or 100 wt % of the amylase content in the enzymatic formulation. In some embodiments, the amount of α-amylase can be at least 10 wt %, at least 20 wt %, at least 30 wt %, at least 40 wt %, at least 50 wt %, at least 60 wt %, at least 70 wt-%, at least 80 wt %, at least 90 wt %, or 100 wt % of the non-proteolytic enzymatic component. In some embodiments, the amount of α-amylase can be at least 10 wt %, at least 20 wt %, at least 30 wt %, at least 40 wt %, at least 50 wt %, at least 60 wt %, at least 70 wt-%, at least 80 wt %, at least 90 wt %, or 100 wt % of the enzymatic formulation.

In some embodiments, the remaining portion of the non-proteolytic enzymatic component (20 wt % or less, 15 wt % or less, 10 wt % or less, 5 wt % or less, 1 wt % or less, 0.5 wt % or less in the enzymatic formulation) can be, but not limited to, hydrolytic, lytic, and oxidative/reductive enzymes including, but are not limited to, hydrolytic, lytic, and oxidative/reductive enzymes selected from the group consisting of lipases, hyaluronidases, chondroitinases, heparanases, heparinases, peroxidases, xylanases, nucleases, phospholipases, esterases, phosphatases, isoamylases, maltases, glycosylases, galactosidases, cutinases, lactases, inulases, pectinases, mannanases, glucosidases, invertases, pectate lyases, reductases, oxidases, phenoloxidases, lipoxygenases, ligninases, pullulanases, tannases, pentosanases, glucanases, arabinosidases, sulfatases, cellulases, hemicellulases, laccases, mixtures thereof, and the like.

In some embodiments, the non-proteolytic component is greater than a proteolytic component in the enzymatic formulation. In some embodiments, a ratio of non-proteolytic enzymes to proteolytic enzymes in the enzymatic formulation is at least 10:1, at least 20:1 at least 40:1, at least 60:1, at least 80:1, or at least 100:1. When the amount of proteolytic enzymes is 0 and the amount of non-proteolytic enzyme is greater than 0, the ratio is ∞: 1 (i.e., greater than 10:1 or any of the other recited ratios). In some embodiments, the enzymatic formulation comprises less than 0.01% by weight proteolytic enzymes, or less than 0.001% by weight of proteolytic enzymes, based on the total weight of enzymes in the enzymatic formulation. In some embodiments, the enzymatic formulation comprises up to 10% by weight of proteolytic enzyme, or up to 5% by weight of proteolytic enzyme, or up to 1% by weight of proteolytic enzyme. Examples of proteolytic enzymes that can be present in the enzymatic formulation include, but are not limited to, proteases and keratinases.

The composition can include an aqueous media. In some embodiments, the aqueous media can have a pH in the range 3.0-10.0, or from 4.5-8.0, or from 5.5 to 7.5. Where the composition is an aqueous-based solution, gel or paste, a water-soluble polymer can be added to increase solution viscosity and to prolong residence time of the enzymatic formulation on and within the denatured collagen-containing material.

In some aspects, a method of preparing collagen peptides is provided. The method can include forming an amylase-containing composition of any embodiment described herein; and contacting the denatured collagen with the enzymatic formulation.

In some embodiments, the method comprises a denaturing step, comprising denaturing a collagen-containing material to form the denatured collagen. In some embodiments, the denaturing step comprises contacting a collagen-containing material with heat, a basic solution, an acidic solution, or combinations thereof. The denaturing step can be conducted using a starting material including mammalian, marine, avian tissues, or combinations thereof.

In some embodiments, the denatured collagen-containing material comprises at least one of the following: denatured bovine collagen, denatured porcine collagen, denatured murine collagen, denatured avian collagen, denatured marine collagen, or combinations thereof.

In some embodiments, the enzymatic formulation and denatured collagen-containing material are in contact for a period ranging from 15 minutes to 48 hours. In some embodiments, contact can last for at least 30 minutes, or at least 45 minutes, or at least 1 hour. In some embodiments, contact can last for up to 24 hours, or up to 12 hours, or up to 8 hours, or up to 4 hours, or up to 2 hours.

In some embodiments, the method includes a deactivating step, whereby the amylase is deactivated. In some embodiments, the deactivating step occurs after the contacting step. In some embodiments, the deactivating step comprises a heat treatment.

In some embodiments, the method includes a second contacting step, comprising contacting the denatured collagen-containing material with a second enzymatic formulation. In some embodiments, the second enzymatic formulation has a composition of any of the enzymatic formulations described herein.

In some embodiments, the composition provides for the enzymatic formulation to be applied on the surface of the denatured collagen-containing material to digest the material to form hydrolyzed collagen peptides. In some embodiments, the enzymatic formulation and the denatured collagen-containing material can be in contact for about 15 minutes to 48 hours, 30 minutes to 24 hours, 1 hour to 12 hours, 1 hour to 8 hours, 1 hour to 4 hours, or 1 hour to 2 hours before removal.

In some embodiments, the denatured collagen-containing material may be repeatedly in contact with the enzymatic formulation with or without washing between repeated application of the enzymatic formulation. In some embodiments, the enzymatic formulation in each contacting step can be the same. In some embodiments, the enzymatic formulation in at least two of the contacting steps can be different.

In some embodiments, the composition described herein can include or be in the form of powder, particulates, liquid, gel, hydrogel, foam, paste, spray, film, or combinations thereof.

### EXAMPLES

The following ingredients and their abbreviations are used in the following discussion:

### Enzymes

α-Amylase, Bacillus amyloliquefaciens spp. powder, 776 U/mg, SD80, Amano, lot KSR08U0201K

### Other Ingredients

Bovine skin gelatin, Sigma, lot SLBM7200V

Sliced ET-AT bovine corium, Bovine Collagen Products, lot 240916

Dulbecco's Phosphate Buffered Saline, DPBS, pH 7.1, Sigma Aldrich, D8537, lot RNBC1143.

Water, Deionized, adjusted to pH 7.

### Example 1: Digestion of Bovine Gelatin Using Amylase

Bovine skin gelatin (10 g) was mixed into water (99 g) and heated to 37° C. Once the gelatin was dissolved, amylase (1 g) was added to the solution and the solution stirred for 25 hours at 37° C. The solution was then centrifuged at 10° C. at 2,000 g force for 10 minutes resulting in a clear yellow solution. To confirm that digestion of the gelatin had occurred, an aliquot was taken to room temperature. No gelation or precipitation occurred as would happen with untreated gelatin. Thus, the digested solution was lyophilized to produce an off-white powder.

This experiment was repeated using 0.5 grams of amylase (half the above amount), which also resulted in an off-white powder.

In both Example 1 experiments, the resulting hydrolyzed collagen had a molecular weight range of up to 5,000 Da, and more than 75% of the hydrolyzed collagen was at 2,500 Da or less.

### Example 2: Digestion of Bovine Corium Using Amylase

Sliced bovine corium, pretreated with the enzymatic treatment/alkaline treatment (ET/AT process), was purchased in “rice grain” size. Standard ET-AT processes were utilized for endotoxin and viral inactivation. The pre-treated corium (21.82 g) was dispersed in 100 ml of buffered pH 8.14 solution and placed into a 90° C. oven for 16 hours at which point the corium was denatured and had become solvated into a translucent aqueous solution. The solution was cooled to 60° C. and amylase (0.21 g) was added and stirred for 100 minutes. A 1 mL aliquot was taken to confirm that no gel formed upon cooling to room temperature. Amylase was deactivated by heating the aliquot to 90° C. for 80 minutes, during which time visible precipitate (amylase) was observed. Following centrifugation to remove the precipitate, the hydrolyzed collagen solution was lyophilized resulting in a white powder. The molecular weight of the hydrolyzed collagen was up to 5,000 Da with >75% of the peptides below 2,500 Da.

### Example 3: MTT Data with Hydrolyzed Collagen

Hydrolyzed collagen produced in Example 2 was compared to commercially available hydrolyzed collagen in a primary human fibroblast proliferation assay.

Primary human dermal fibroblasts (HDFa, PCS-201-012, P5) were cultured to 100% confluency in a T75 flask using fibroblast basal medium. The cells were detached and resuspended in 2% low serum medium containing penicillin, streptomycin, and amphotericin B to prevent infection. They were then seeded at N=3 for each formulation and control. Then, 0.5 mL of the cell suspension was added to each well. The plate was incubated for 24 hours at 37° C. The preparations were prepared at 1.75% w/v in serum-free fibroblast medium supplemented with antibiotics (penicillin, streptomycin, and amphotericin B). Once the media was removed, 1 mL of each solution was added to the seeded wells. The plates were then placed back in the incubator for 24 hours. The next day, MTT (3-(4,5-dimethylthiazol-2-yl)-2,5-diphenyltetrazolium bromide) solution was used, and 1 mL of this solution was added to each well. The plate was incubated for 4 hours at 37° C. After incubation, the medium was removed from each well, and 1 mL of solubilizing solution (dimethyl sulfoxide (DMSO)) was added to each well. Next, 200 μL from each well of the 24-well plate was transferred to a corresponding well in a 96-well plate. The plate reader then measured the absorbance of each well at 540 nm. The data were normalized using the 2% fibroblast basal media as control and calculated as percentage of control.

Hydrolyzed collagen produced in Example 2 had comparable cell proliferation to commercially available hydrolyzed collagen.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. An amylase-containing composition, comprising:
denatured collagen; and
an enzymatic formulation comprising at least 50 wt-% of amylase based on the weight of total solids in the enzymatic formulation.

2. The amylase-containing composition of claim 1, wherein the enzymatic formulation comprises at least 50 wt-% of α-amylase.

3. The amylase-containing composition of claim 1, wherein the enzymatic formulation comprises α-amylase, β-amylase, γ-amylase, or combinations thereof.

4. The amylase-containing composition of claim 1, wherein the enzymatic formulation comprises a weight ratio of non-proteolytic enzymes to proteolytic enzymes of at least 10:1.

5. The amylase-containing composition of claim 1, wherein the enzymatic formulation comprises less than 0.1 wt-% of proteolytic enzymes based on the total solids in the enzymatic formulation.

6. The amylase-containing composition of claim 1, wherein the denatured collagen comprises one or more of denatured bovine collagen, denatured porcine collagen, denatured murine collagen, denatured avian collagen, denatured marine collagen or combinations thereof.

7. The amylase-containing composition of claim 1, comprising a keratolytic agent.

8. The amylase-containing composition of claim 7, wherein the keratolytic agent is selected from the group consisting of urea, salicylic acid and α-hydroxyacids, such as lactic acid, glycolic acid, citric acid, and combinations thereof.

9. The amylase-containing composition of claim 1, comprising a keratolytic agent in an amount of up to 15 wt-% of solids in the composition.

10. The amylase-containing composition of claim 1, comprising a surfactant in an amount up to 10 wt % based on the solids in the composition

11. The amylase-containing composition of claim 1, wherein the amylase-containing composition is an aqueous composition with a pH in the range of 6.5 to 7.5.

12. The amylase-containing composition of claim 1, wherein the amylase-containing composition is an aqueous composition with a pH in the range of 3.0 to 6.5.

13. The amylase-containing composition of claim 1, wherein the amylase-containing composition is an aqueous composition with a pH in the range of 7.5 to 10.

14. The amylase-containing composition of claim 1, comprising a penetration enhancer in an amount up to 5 wt % based on the solids in the composition.

15. The amylase-containing composition of claim 1, wherein a weight ratio of denatured collagen to enzymatic formulation ranges from 100:1 to 10:1.

16. A method of preparing collagen peptides, comprising:
forming an amylase-containing composition of claim 1; and
contacting the denatured collagen with the enzymatic formulation, comprising at least 50 wt-% amylase based on the weight of total solids in the enzymatic formulation, wherein the resulting collagen peptides have a molecular weight of less than 10,000 Da.

17. The method of claim 16, further comprising a denaturing step, comprising denaturing a collagen-containing material to form the denatured collagen.

18. The method of claim 17, wherein the collagen-containing material is from a source selected from mammalian, avian, marine, and combinations thereof.

19. The method of claim 17, wherein the denaturing step comprises exposing the collagen-containing material to heat, a basic solution, an acidic solution, or a combination thereof.

20. The method of claim 16, wherein the contacting step comprises contacting the denatured collagen with the enzymatic formulation for a period ranging from 15 minutes to 48 hours.

21. The method of claim 16, further comprising a deactivating step, whereby the amylase is deactivated.

22. The method of claim 16, further comprising a second contacting step, comprising contacting the denatured collagen, which has already been contacted with the enzymatic formulation, with a second enzymatic formulation.
